(19) United States
(12) Patent Application Publication
(10) Pub. No.: US 2024/0383838 A1
(43) Pub. Date: Nov. 21, 2024

(54) Synthesis of muconic acid (ester) from aldaric acid (ester)

(71) Applicant: Teknologian tutkimuskeskus VTT Oy, Espoo (FI)

(72) Inventors: Nicolaas Van Strien, Espoo (FI); Sari Rautiainen, Espoo (FI)

(21) Appl. No.: 18/686,889

(22) PCT Filed: Aug. 26, 2022
(86) PCT No.: PCT/FI2022/050551

§ 371 Date: Feb. 27, 2024

(30) Foreign Application Priority Data
Aug. 27, 2021 (FI) 20215901

(51) Int. Cl. C07C 67/327; C07C 51/09

(52) U.S. Cl. CPC C07C 67/327; C07C 51/09

(57) ABSTRACT

According to an example aspect of the present invention, there is provided an energy efficient and environmentally benign method for producing muconic acid and muconic acid esters from aldaric acid esters.

### FIELD

The present invention relates to a method for producing muconic acid (ester) from aldaric acid (ester) by using methyl acetate as a reaction solvent.

### BACKGROUND

Muconic acid (IV) and muconic acid esters (V, VI) may be produced via the hydrodeoxygenation of aldaric acids (I). Aldaric acids, such as galactaric acid or glucaric acid, can be produced from pectin, starch and other carbohydrates both edible and non-edible. By converting aldaric acids to muconic acid, a doorway is opened which allows for a wide variety of compounds to be prepared from bio-based resources, which would otherwise be prepared from crude oil stock. WO 2015/189481 describes the production of sugar acid platform chemicals, more precisely muconic acid, from aldaric acid(s) via selective catalytic hydrodeoxygenation. This method can be efficient, but it requires a high dilution and catalyst concentration. The resultant product is typically afforded in low to good yield (17-70%), but the methyltrioxorhenium catalyst (MTO) is highly expensive and catalyst reuse is a challenge.

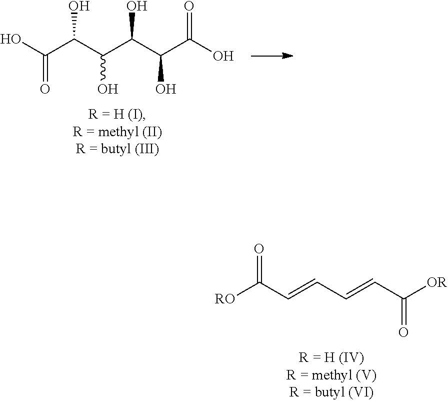

Biotechnically muconic acid can be produced via micro-organisms, but the yield is limited to approximately 35% due to the efficiencies inherit in using micro-organisms that also require carbohydrate feedstock.

Continuous production of muconic acid from saccharic acid using ammonium perrhenate is also possible (WO 2017/207875), however, this is carried out by using an alternative aldaric acid (glucaric acid) and a continuous flow reactor (CFR) rather than a batch reactor. The CFR has, unlike the batch reactor, a low dilution, low contact time between reagents and catalysts and it is also run at a lower temperature. The batch reactor process uses a higher concentration for a predetermined amount of time and at a set temperature where there is greater contact time between reagents and catalyst.

WO 2019/155128 on the other hand describes a method for producing muconic acid ester from aldaric acid ester, and for separating and purifying the produced muconic acid ester by high vacuum distillation in a total heating environment. In this method, ammonium perrhenate is used as a reaction catalyst. This catalyst can be readily filtered from the reaction mixture, which gives the potential for the reuse of catalyst and keeps the reactor cleaner after the reaction thus avoiding heavy mechanical cleaning. Also this method, however, requires a high dilution and catalyst concentration.

There is a need for a novel technology, which focuses on finding a cheaper and more energy efficient solvent than for example n-butanol or methanol. This reduces the energy costs during the purification step of the process. Using a reaction solvent which doesn't undergo dehydration with the use of hydrogen donating rhenium catalysts at used temperatures, will also decrease the solvent loss and will increase the product yield. The original patent route using mucic acid can produce muconic acid and muconic acid ester in good yield, however, even with a good conversion the reactor contains decomposed detritus after the reaction, which is fixed/adhered to the reactor wall. This is difficult to clean and could therefore be a limiting factor when scaling-up the process to run at an industrial scale. It is important to develop cheap, efficient and environmentally benign synthesis process of muconic acid. Avoiding use of expensive catalysts and solvents is essential.

### SUMMARY OF THE INVENTION

The invention is defined by the features of the independent claims. Some specific embodiments are defined in the dependent claims.

According to an aspect of the present invention, there is provided a method for producing muconic acid (ester) from aldaric acid (esters) by using methyl acetate as a reaction solvent.

This and other aspects, together with the advantages thereof over known solutions are achieved by the present invention, as hereinafter described and claimed.

The method of the present invention is mainly characterized by what is stated in the characterizing part of claim 1.

Considerable advantages are obtained by means of the invention. For example, the method described herein uses more soluble mucic acid ester form to improve yield and reduce the solvent use. Methyl acetate solvent is considered greener solvent with lower boiling point than used solvents in prior art solutions. The change of solvent has been proved to increase the production yield substantially. Other benefits with the use of methyl acetate are for example: a) retaining the solvent due to the significantly reduced formation of dimethyl ether, which occurs in higher amounts with methanol, b) a unique solvent, which has not been previously used in the synthesis of muconic acid, and c) the reaction can be achieved in 4 h rather than previously reported over 24 h, giving substantial energy savings.

Next, the present technology will be described more closely with reference to certain embodiments.

### EMBODIMENTS

The present technology provides improved and cost-efficient synthesis method of muconic acid (ester) from aldaric acid (esters) by using a bio-based non-alcoholic reaction solvent, preferably methyl acetate, and a suitable catalyst in a pressurized reactor conditions.

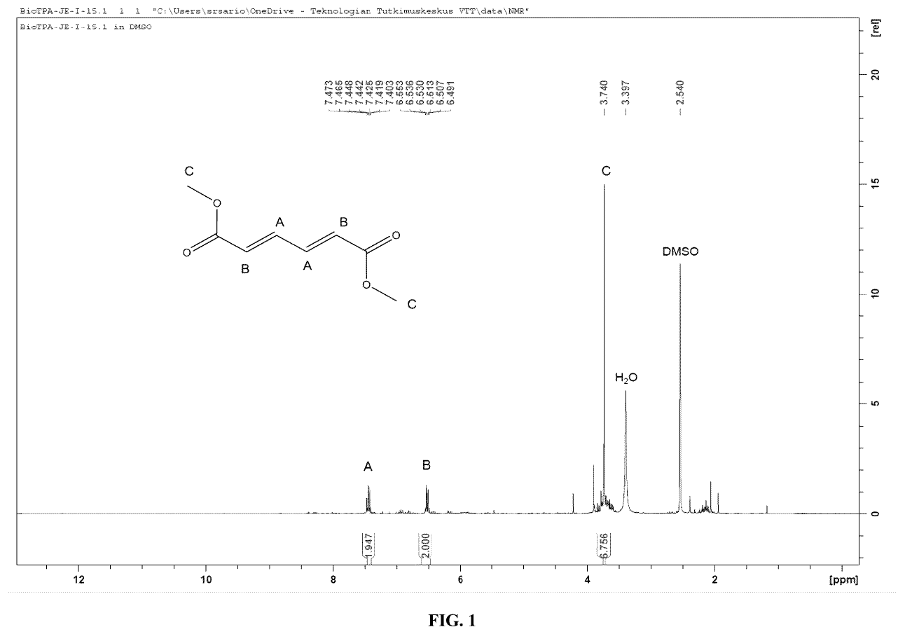

FIG. 1 is an NMR spectrum of the produced muconic methyl ester.

According to an embodiment of the present invention, the method for producing muconic acid ester comprises at least the steps of:
adding an aldaric acid ester and a catalyst into a pressure reactor,
adding a bio-based non-alcoholic solvent to the reactor,
pressurizing the reactor to overpressure with an inert gas,
increasing the temperature inside the reactor between 175° C. and 200° C. and mixing the content for a pre-determined reaction time,
cooling the reactor to room temperature of 20 to 25° C.,
filtering the catalyst and removing the solvent by evaporation, and
collecting the formed product.

According to one embodiment of the present invention, the aldaric acid ester is mucic acid ester.

According to one embodiment of the present invention, the catalyst is a rhenium catalyst. More precisely, it is herein preferred to use ammonium perrhenate as the catalyst. By using rhenium catalysts, such as ammonium perrhenate, the amount of catalyst is drastically reduced compared to the existing technology, which uses methyltrioxorhenium catalyst, which is typically over 100-times more expensive.

According to one embodiment of the present invention, it is preferred to use 20 to 30 mol-% catalyst loading, such as about 23 mol-%, when reaction time of 24 hours is applied. When the reaction time is set to 4 hours, it is preferred to use 10 to 20 mol-% catalyst loading, such as about 14 mol-%.

According to one embodiment of the present invention, the solvent is acetic acid ester or formic acid ester, preferably methyl acetate. The use of methyl acetate enables the use of the methyl ester of the starting material. With the use of methyl acetate as a reaction solvent, the problems relating to formation of dimethyl ether when using methanol solvent (as in the existing technology), is reduced. In addition, methyl acetate has not been shown to date to be used in the synthesis of muconic acid (ester). Furthermore, methyl acetate is a cheap reaction solvent that can be easily removed from the reaction mixture due to its low boiling point. It has also lower health risks compared to methanol or n-butanol.

According to one embodiment of the present invention, the reaction is carried out in a pressure reactor, such as in a Hastelloy pressure reactor. The substrate and catalyst are added to the reactor followed by solvent. The reactor is then pressurized to overpressure, such as to at least 5 bars, with an inert gas, for example nitrogen. The temperature is increased up to 200° C. and the contents are stirred for 4 hours, or up to 175° C. and stirred for 20 hours, before cooling to room temperature. The catalyst is then filtered away and the solvent removed by evaporation. The brown-black solid isolated is crude product muconic methyl ester.

Thus, according to one embodiment of the present invention, the reaction is carried out during 4-hour reaction time. Existing synthesis methods for muconic acid typically requires at least 24-hour reactions, whereby running the reaction for 20-hours shorter saves significant amount of energy and provides improvements to the techno-economic assessment of the production process. It is thus preferred that the reaction time is set between 4 hours to 24 hours, preferably between 4 hours to 20 hours, such as 4 hours or in any case up to 24 hours.

One further advantage of the present invention is that the muconic acid (ester) synthesis route disclosed herein produces fewer side-products than previously reported. Having fewer side-products benefits the downstream processing.

Reference throughout this specification to one embodiment or an embodiment means that a particular feature, structure, or characteristic described in connection with the embodiment is included in at least one embodiment of the present invention. Thus, appearances of the phrases “in one embodiment” or “in an embodiment” in various places throughout this specification are not necessarily all referring to the same embodiment. Where reference is made to a numerical value using a term such as, for example, about or substantially, the exact numerical value is also disclosed.

As used herein, a plurality of items, structural elements, compositional elements, and/or materials may be presented in a common list for convenience. However, these lists should be construed as though each member of the list is individually identified as a separate and unique member. While the forgoing examples are illustrative of the principles of the present invention in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the invention be limited, except as by the claims set forth below.

The verbs “to comprise” and “to include” are used in this document as open limitations that neither exclude nor require the existence of also un-recited features. The features recited in depending claims are mutually freely combinable unless otherwise explicitly stated.

### INDUSTRIAL APPLICABILITY

At least some embodiments of the present invention find industrial application in generating a full value chain from the forest industry, agriculture, or food industry side streams to platform chemicals and end applications. In principle, this chain comprises production of aldaric acids from aldoses and side-stream carbohydrates, converting the aldaric acids to dicarboxylic acids, which in turn are used as platform chemicals for various bio-based applications, such as bio-based polyesters and nylon. Thus, according to one example, the present method produces muconic acid for use in the production of polyesters, polyamides and PET co-monomers.

### EXAMPLES

### Set 1: Methyl Acetate with Reaction Time of 24 h

Mucic acid methyl ester (0.239 g, 1.0 mmol) was added to a Hastelloy C-276 pressure reactor. To this was then added ammonium perrhenate (0.23 mmol, 22.8 mol %) and methyl acetate solvent. A stirrer bar was added and the reactor was then sealed and flushed with nitrogen before pressurising to approximately 5 bar. The reactor was then heated to the required temperature and stirred for a specific time. Once the reaction was completed, the reactor was cooled to room temperature and the contents removed. Vacuum filtration and evaporation of solvent (40° C., below 10 mbar) afforded the product as a solid. The reaction product was purified by using known technology and was characterized GC-MS and ¹H NMR. Yields are interpreted from GC-FID.

TABLE 1

| | Reaction conditions | | | | | Purified yield |
|---|---|---|---|---|---|---|
| | Catalyst | Solvent | | | Mass | Muconic acid |
| | (mol- | volume | Temperature | Time | isolated | dimethyl ester |
| Entry | %) | (cm³) | (° C.) | (h) | wt-% | mol-% |
| 1 | 22.8 | 15 | 175 | 24 | 99 | 63.2 |
| 2 | 22.4 | 15 | 150 | 24 | 96 | 22.5 |
| 3 | 22.4 | 15 | 200 | 24 | 35 | <1 |

### ¹H-NMR (DMSO-d₆, 500 MHz)

Muconic acid dimethyl ester: δ=7.442; (dd, 2H, CH), 6.520; (dd, 2H, CH), 3.740; (s, 6H, CH₃)

### Set 2: methyl acetate with reaction time of 4 h

Mucic acid methyl ester (0.385 g, 1.6 mmol) was added to a Hastelloy C-276 pressure reactor. To this was then added ammonium perrhenate (0.22 mmol, 13.8 mol %) and methyl acetate solvent. A stirrer bar was added and the reactor was then sealed and flushed with nitrogen before pressurising to approximately 5 bar. The reactor was then heated to the required temperature and stirred for a specific time. Once the reaction was completed, the reactor was cooled to room temperature and the contents removed. Vacuum filtration and evaporation of solvent (40° C., below 10 mbar) afforded the product as a solid. The reaction product was purified by using known technology and was characterized GC-MS and ¹H NMR. Yields are interpreted from GC-FID.

TABLE 2

| | Reaction conditions | | | | | Purified yield |
|---|---|---|---|---|---|---|
| | | Solvent | | | Mass | Muconic acid |
| | Catalyst | volume | Temperature | Time | isolated | di methyl ester |
| Entry | (mol-%) | (cm³) | (° C.) | (h) | wt-% | mol-% |
| 4 | 13.8 | 15 | 175 | 4 | 78 | 28.5 |

¹H-NMR (DMSO-d₆, 500 MHz)

Muconic acid dimethyl ester: δ=7.442; (dd, 2H, CH), 6.520; (dd, 2H, CH), 3.740; (s, 6H, CH₃).

### CITATION LIST

### Patent Literature

WO 2015/189481
WO 2017/207875
WO 2019/155128

1. A method for producing muconic acid and muconic acid esters, wherein the method comprises at least the steps of:
adding a mucic acid ester and a rhenium catalyst into a pressure reactor, and by applying a catalyst loading of from 10 to 30 mol-%.
adding a bio-based non-alcoholic solvent to the reactor, wherein the bio-based non-alcoholic solvent comprises an acetic acid ester or a formic acid ester,
pressurizing the reactor to overpressure with an inert gas,
increasing the temperature inside the reactor between 175° C. and 200° C. and mixing the content for a pre-determined reaction time of 4 to 24 hours,
cooling the reactor to room temperature of 20 to 25° C.,
filtering the catalyst and removing the solvent by evaporation, and
collecting the formed product.

2. (canceled)

3. (canceled)

4. The method according to claim 1, wherein the rhenium catalyst is ammonium perrhenate.

5. The method according to claim 1, characterized in that wherein the reaction time is between 4 hours and 24 hours, preferably between 4 hours and 20 hours, and most suitably about 4 Hours.

6. The method according to claim 1, wherein the catalyst loading is from 20 to 30 mol-%, when the reaction time is 24 hours.

7. The method according to claim 1, wherein the catalyst loading is from 10 to 20 mol-%, when the reaction time is 4 hours.

8. The method according to claim 1, the bio-based non-alcoholic solvent comprises methyl acetate.

9. The method according to claim 1, wherein the reactor is pressurized with nitrogen gas to at least 5 bars.

10. The method according to claim 1, wherein the temperature inside the reactor is increased to 200° C., when the reaction time is 4 hours.

11. The method according to claim 1, wherein the temperature inside the reactor is increased to 175° C., when the reaction time is 24 hours.
